(19) United States
(12) Patent Application Publication
(10) Pub. No.: US 2012/0295969 A1
(43) Pub. Date: Nov. 22, 2012

(54) Novel Muscle Relaxant Using Negatively Charged Gold with Choline

(76) Inventor: Chur Chin, Seoul (KR)

(21) Appl. No.: 13/558,722

(22) Filed: Jul. 26, 2012

Related U.S. Application Data
(63) Continuation-in-part of application No. 12/531,875, filed on Sep. 17, 2009.

(30) Foreign Application Priority Data
Jun. 25, 2007 (KR) 10-2007-0061943

(51) Int. Cl. A61K 31/28; A61P 21/02; A61P 9/06; C07F 1/12

(52) U.S. Cl. 514/495; 556/110

(57) ABSTRACT

In order to achieve muscle relaxing effect and anti-atrial fibrillation effect, the present invention provides gold nanoparticles complexes. Gold nanoparticles complexes include negatively charged gold nanoparticles bound to choline or spermidine through ionic bonds according to the purpose to be achieved, which form gold nanoparticles-choline complexes or gold nanoparticles-spermidine complexes. The complexes migrate to an acetylcholine receptor and then the ionic bonds of the complexes are broken, thereby inducing gold-sulfur bonds in the Cys loop of an acetylcholine receptor, thus block the ionic path and achieve the effects.

### CROSS REFERENCE TO RELATED APPLICATIONS

This patent application is a continuation-in-part application of U.S. patent application Ser. No. 12/531,875, filed Sep. 17, 2009, which is incorporated herein by reference in its entirety.

### BACKGROUND OF THE INVENTION

1. Field of the Invention

Choline is a kind of vitamin B complex, and acts as a partial agonist for acetylcholine, a neurotransmitter, in neuromuscular junction. Negatively charged gold particles are formed by adhering alkaline COO⁻ particles to a thin film of gold nanoparticles. When the negatively charged gold particles form ionic bonds with choline and then the resulting complexes are injected intravenously, the complexes migrate to an acetylcholine receptor in neuromuscular junction of a patient within 1 minute, and the choline binds to an acetylcholine-binding site thereby forming ionic bonds. The migrated nanoparticles enter the extracellular vestibule of the receptor together with a high concentration of the choline and migrate to a cysteine loop to thereby bind to the SH groups of the cysteine due to the strong binding force of gold-sulfur ions, and subsequently prevent from forming alpha subunit S═S bonds thereby blocking a flow of electric current through open ion gates. Accordingly, it is anticipated that the choline-gold nanoparticle complexes will perform a role of blocking the signal transmission in neuromuscular junction more strongly. Spermidine is a kind of polyamine and act as a blocking agent of inward rectifying potassium channels in human atrial cells. Spermidine, which is binding to 3 molecules of gold nanoparticles can introduce into the ion pore.

Since the action of cholinesterase is inhibited if reverse is administered, acetylcholine is not degraded and the acetylcholine bound to the receptor induces S═S reducing bonds in the cysteine loop. The negatively charged gold nanoparticles are come out of the ionic paths by repulsion since the gold-sulfur binding force is released due to the electric current generated by the reducing bonds.

2. Description of Related Art

The signal transduction at neuromuscular junction is achieved by chemical means such as a neurotransmitter, or physical means such as an ionic path. Prior muscle relaxant is chemical means that blocks neuromuscular transmission since it has a similar structure to acetylcholine, a neurotransmitter, and thus binds competitively to acetylcholine and its receptor. If a method of blocking the flow and maintenance of electric current at ionic paths by using gold nanoparticles having a negative charge is used in combination with the prior means, the effect of muscle relaxing will be doubled. Further, such strong muscle relaxing effect can be applied as an anticonvulsant.

### SUMMARY OF THE INVENTION

### Choline

Choline is a vitamin B complex having 2-hydroxy-N,N,N-trimethylethanaminium structure, and passes through the cerebrovascular barriers. It takes only 1-2 msec for acetylcholine isolated from a presynaptic terminal to work on the postsynaptic membrane. Once a postsynaptic stimulant voltage is generated, acetylcholine is hydrolyzed into choline and acetate by cholinesterase. The choline and acetate are re-synthesized into acetylcholine by choline acetylase and are used.

### Spermidine

The polyamines putrescine, spermidine and spermine are present in almost all cells and have important roles in protein synthesis, cell division and cell growth. Polyamines are organic polycations that are protonated at physiological pH and can potentially interact with a variety of cellular targets including nucleic acids and proteins.

### Signal Transduction at Neuromuscular Junction

A neurotransmitter in a synaptic vesicle is released by a nerve impulse transmitted from a cell body to an axon terminal. As the nerve impulse reaches a cell membrane of a terminal bouton along the axon, a calcium (Ca⁺⁺) channel present in the location is opened, and calcium ions enter the terminal bouton, and the membrane of the synaptic vesicle is fused with the cell membrane.

When a presynaptic cell membrane is fused to the synaptic vesicle, the neurotransmitter in the synaptic vesicle is released into synaptic clefts and binds to a receptor present in a postsynaptic cell membrane. The receptor acts as a kind of a ligand-sensitive ion channel. The channel is an ion channel that is opened by coupling with a ligand, i.e., the neurotransmitter. When a cation channel such as a sodium channel or a calcium channel is opened, a cation that was present in extracellular fluid enters the postsynaptic portion, and thus the voltage inside the cell that has maintained a stable membrane electric potential (−80 mV) is partially changed. As the voltage reaches a threshold (−70 mV), a voltage-sensitive sodium channel is opened and a large quantity of the sodium ions in the extracellular fluid come inside the cell, and accordingly depolarization occurs thereby reversing the membrane electric potential of this portion. As the membrane electric potential is changed like this, a voltage sensitive potassium channel is now opened. As potassium ions come outside the cell, the membrane electric potential returns to its original status.

The sodium and potassium ions in which the outside and inside of the cell are reversed from the original status are exchanged in 1:1 ratio by a sodium/potassium (Na⁺/K⁺) exchange pump. At this time, energy is needed.

When the ion exchange is completely finished, the membrane electric potential returns to the original status and the membrane again becomes capable of responding to a stimulus.

The sodium ions enter a specific portion of a cell and depolarize not only the portion but also are diffused along an axon and depolarize the adjacent portion. If the diffusion occurs toward a cell body, the sodium channel is inactivated and thus an impulse is not continuously transmitted. However, if the sodium ions are diffused toward the synaptic terminal of the axon, the adjacent portion of the membrane is depolarized and the active electric potential is also generated at this portion.

### Human Inward Rectifier Potassium Channels in Atrial Fibrillation

The inward rectifier K currents are increased in atria of patients with Atrial fibrillation. Atrial fibrillation (AF) is characterized by pacing model. Vagal stimulation causes bradycardia and decreases contractility via activation of another inward rectifier current, the acetylcholine cativated K current.

### Structure of an Acetylcholine Receptor

A nicotinic acetylcholine receptor is an ionic receptor controlling the ionic path in a cell membrane through a ligand. The nicotinic acetylcholine receptor consists of five subunits, and each subunit is very similar in structure. An acetylcholine receptor of a muscle consists of two a subunits and β, γ and δ subunits. The site binding with the receptor in a muscle is outside a subunits near N termini. If acetylcholine binds to the site, the subunits become more similar to other subunits in structure, and thus the ionic path becomes more symmetrical and is opened to a size of about 0.65 nm.

The nicotinic acetylcholine receptor has a similar structure to a muscle receptor on signal transduction in a nerve. When acetylcholine binds continuously to the site, the ionic path maintains its open state. As the ionic path is opened, ions having a positive charge, particularly sodium and calcium ions, enter inside the cell.

Na⁺ and K⁺ are all penetrable to the nicotinic acetylcholine receptor, and in a certain site, Ca²⁺ is also penetrable. Na⁺ and K⁺ penetrate an open site of the ionic path, and this process occurs by the change in the structure of each subunits.

It is known that the nicotinic acetylcholine receptor consists of extracelluar, transmembrane and intracellular domains (ICD) from research through Gaussian Network Model (GNM) and Anisotropic Network Model (ANM). The opening and closing of the ionic path are determined by symmetrical 3-dimensional twisting movement and asymmetrical tilting movement between each subunit. At this time, the pore size between paths is determined by the opening and closing of the disulfide (S═S) bond of cysteine (Cys) loop in and between subunits. Accordingly, if gold nanoparticles having a negative charge interrupt the disulfide bond of Cys loop between each subunit, the flow and maintenance of the electric current to the neuromuscular junction site can be blocked.

The present invention provides gold nanoparticle complexes with choline or spermidine to achieve muscle relaxing effect or anti-atrial fibrillation effect. In one embodiment, the present invention provides a drug having muscle relaxing effect by binding negatively charged gold nanoparticles having a size of 1.4 nm fitted to synaptic clefts to choline through ionic bonds, thereby inducing gold-sulfur bonds in the Cys loop of an acetylcholine receptor in neuromuscular junction, and thus blocking the electric current induced in signal transduction from a nerve to a muscle, thereby blocking neuromuscular transmission chemically and physically. When the reverse is administered after surgery, the gold-sulfur bonds are broken due to the re-flow of the electric current, and thus the gold nanoparticles are again released and metabolized into neuromuscular junction. Further, such muscle relaxing effect can be applied as an anticonvulsant. In another embodiment, the present invention provides a drug having an anti-atrial fibrillation effect can be prepared by binding negatively charged gold nanoparticles having a size of 0.8 nm fitted to spermidine through ionic bonds, thereby inducing gold-sulfur bonds in muscarinic acetylcholine receptor in atrial cardiomyocyte, and thus blocking the inward potassium current. When the reverse is administered, the gold-sulfur bonds are broken due to the re-flow of the electric current, and thus the gold nanoparticles are again released.

### BRIEF DESCRIPTION OF THE DRAWING(S)

FIG. 1 illustrates schematically a structure in which ionic bonds are formed between choline having a positive charge and a nanoparticle having a negative charge;

FIG. 2 illustrates schematically a process that choline injected intravenously after forming ionic bonds with gold nanoparticles having a similar structure to acetylcholine binds to an amino acid of a binding site on an acetylcholine receptor, and the nanoparticles, in which the ionic bonds are disrupted, bind to the SH groups of Cys loop by repulsion to the binding site having a negative charge;

FIG. 3 illustrates the analysis results for choline and nanoparticles by UV spectrophotometry, showing that the light-absorbing configurations of the choline and the nanoparticles were similarly achieved; and

FIG. 4 illustrates the analysis results for choline-nanoparticle complexes by UV spectrophotometry, showing that the ionic bonds between the choline and the nanoparticles were smoothly formed by confirming the intensity and the light-absorbing region of the choline-nanoparticle complexes were more extended than those of the respective single molecules;

FIG. 5 is a graph illustrating the effect of choline-nanoparticle complexes over time in an experiment employing Ach, and schematically depicting a process that immediate reversal is achieved with the receptor inactivation effect of about 75% over time; and

FIG. 6 is a graph illustrating the effect of choline-nanoparticle complexes over time in an experiment employing DMPP, and schematically depicting a process that immediate reversal is achieved with the receptor inactivation effect of about 75% over time.

FIG. 7 illustrates schematically a structure in which ionic bonds are formed between spermidine having a positive charge and a nanoparticle having a negative charge;

FIG. 8 illustrates schematically a process that negatively charged gold nanoparticle can block the inward rectifying potassium channels of human atrial cell by gold-sulfur binding between the gold nanoparticles and the sulfur group of the cysteine group in the transmembraneous portion of the muscarinic (M2) acetylcholine receptor. By spermidine molecules binding ionically with three nanoparticles, the complex can enter the intracellular vestibule of the receptor. Currents evoked by acetylcholine in the receptor can break the bondage between the gold and sulfur groups.

### DESCRIPTION OF THE INVENTION

Firstly, ionic bonds are induced by vortexing and pipetting choline having a positive charge and gold nanoparticles having a negative charge for one hour. The degree of binding is determined through the wavelength analysis by UV VIS spectrophotometry.

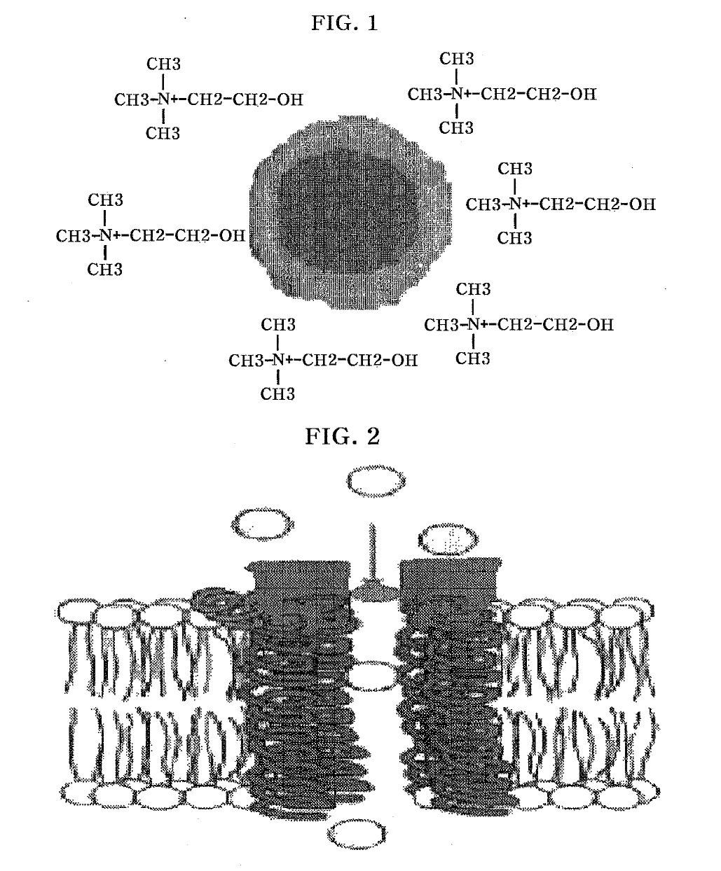

FIG. 1 illustrates ionic bonds between 1 mM choline and 30 nM negatively charged nanoparticle: the configuration that COO⁻ groups coated on a thin film of the nanoparticle binds to N⁺ groups of the choline molecules is schematically illustrated.

FIG. 2 illustrates schematically the binding site of choline and gold nanoparticles having a negative charge on a nicotinic acetylcholine receptor at a neuromuscular junction postsynaptic muscle cell. The red color depicts the extracellular domain of the receptor, the violet color depicts the terminal of the choline molecule binding on the receptor, the blue color depicts transmembrane and intracellular domains, and the pale blue color depicts sodium and calcium ions passing through an ionic path. When choline-nanoparticle Complexes bind strongly to the receptor, the gold nanoparticle molecules are detached from the binding body by repulsion and block the flow of the ionic path through adsorption of the SH groups inside a glycoprotein receptor.

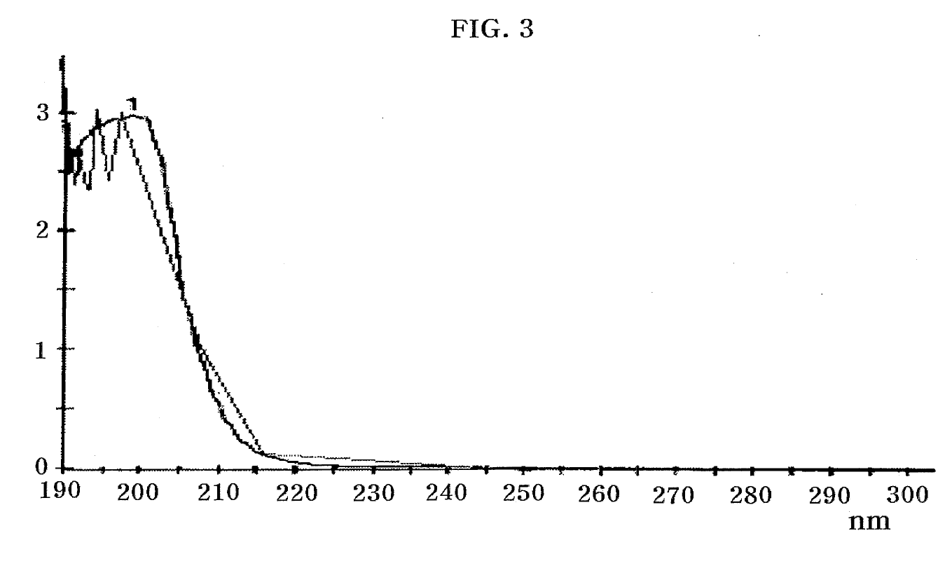

FIG. 3 illustrates the analysis results for choline and gold nanoparticles having a negative charge by UV VIS spectrophotometry. The black graph depicts the analysis results for the choline, and the red graph depicts those for the gold nanoparticles having a negative charge by UV VIS spectrophotometry. It was confirmed that the absorbance of the two molecules was achieved in a similar degree within almost the same wavelength range. The used apparatus was Shimadzu UV3101PC made in Japan. The measurement was made in a light absorbing mode in the UV wavelength range of 190 to 300 nm, and the resolution was possible to 0.1 nm.

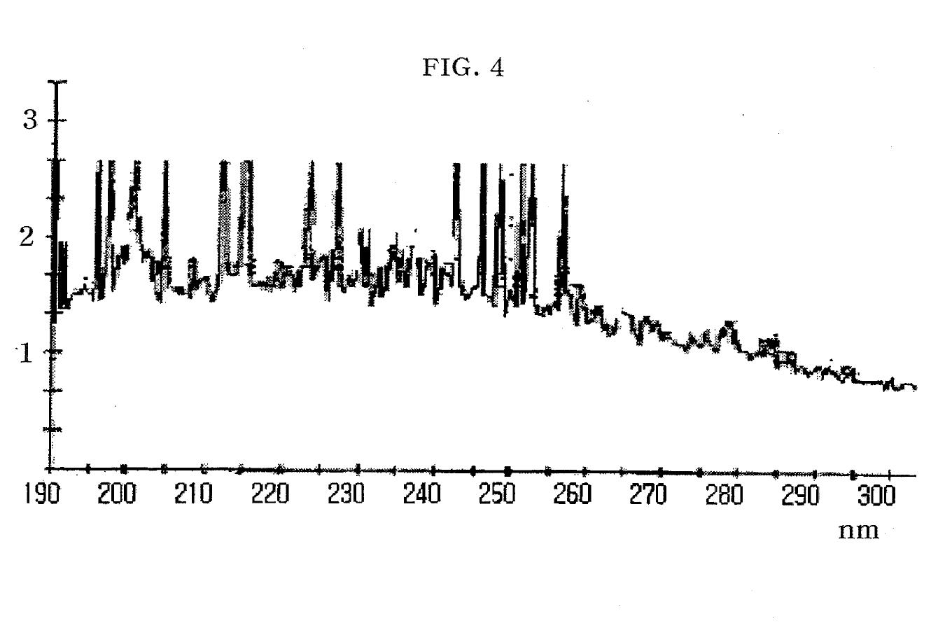

FIG. 4 illustrates the analysis results for complexes of the choline and the gold nanoparticles having a negative charge by UV VIS spectrophotometry, and shows an intensity stronger and a light absorbing range in a wavelength region wider than the absorbance of the respective molecules. The used apparatus was Shimadzu UV3101PC made in Japan. The measurement was made in a light absorbing mode in the UV wavelength range of 190 to 300 nm, and the resolution was possible to 0.1 nm.

The inventor studied the characteristics of cardiac IK1 in freshly isolated adult human atrial myocytes by using the patch-clamp technique. Specimens were obtained from the atria of patients undergoing cardiac surgery. Whole-cell IK1 in atrium exhibited after treated intracellulary 0.8 nm negatively charged gold nanoparticles with 100 mM spermidine ion complexes, the inward current activated by Ach (30 uM) at −100 mV was reduced by 75.4+/−4.4% (n=5). The addition of Ach (30 uM) reversed block and current was 67.9+/−2.9% of control. Further, we found that with the perfused external solution containing the complex, the complex blocked the Ach-activated current by 48.8+/−3.1%. It indicates that even treating extracellularly, it is possible to achieve 50% blocking by cell penetration.

### Organ Model Experiment

The left adrenal of a Sprague-Dawley white rat was removed and cannulated according to the Wakade method, and then it was extirpated and immobilized to a leucite chamber, and an experiment was performed while perfusing with Krebs fluid.

A single injection of ACh (5.32×10⁻³ M) was treated in 0.05 ml of a buffer solution containing 137.5 mM NaCl, 2.5 mM KCl, 2 mM CaCl₂, 1 mM MgCl₂ and 10 mM D-glucose at room temperature for 3 minute. Then, the amperometric current generated by the secreted catecholamine was measured by employing an 8-μm-diameter carbon electrode. Data was computerized with IGOR software and the buffer solution was perfused every two seconds.

The agonistic effect of the choline was confirmed with a control experiment employing 1,1-dimethyl-4-phenylpiperazinium iodide (DMPP), the choline-nanoparticle complexes were treated again for 3 minutes, and then the change in the catecholamine secretion was measured.

Finally, 10 μM DMPP stimulation was performed for 2 seconds as reverse, thereby confirming whether catecholamine secretion was restarted, and verifying whether the choline-nanoparticle complexes were released from the ionic path.

### Result

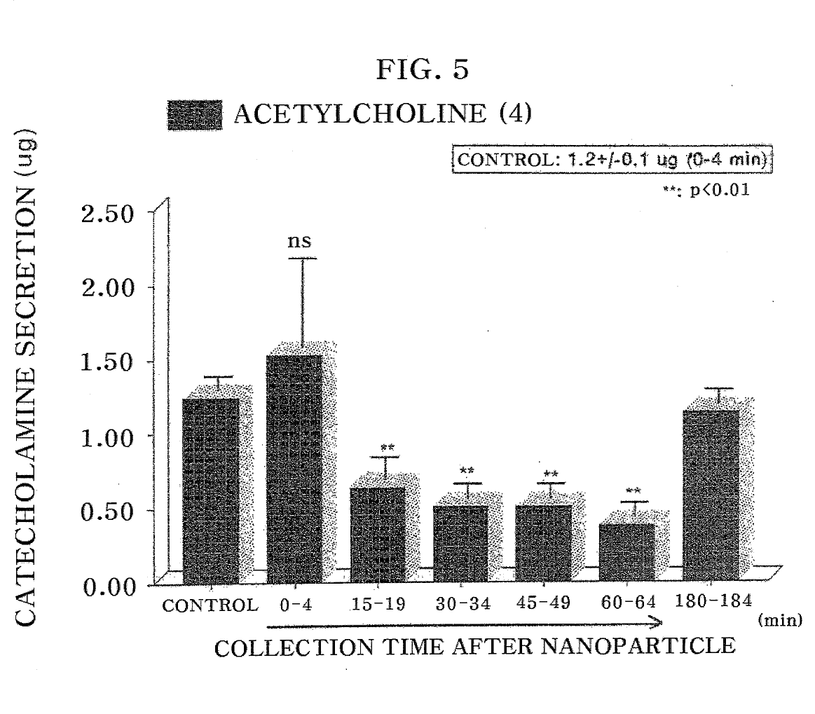

FIG. 5 illustrates the effect of the choline-nanoparticle complexes over time. When 0.05 ml of a single injection of ACh (5.32×10⁻³ M) was evoked in a rat adrenal gland, the receptor activation was confirmed after 15 minutes, and then when the choline-nanoparticle complexes were treated, it was confirmed that the catecholamine secretion was decreased to about 75% after 60 minutes. When Ach was again treated after about 1.5 hours, it was confirmed that immediate reversal was achieved. *: P<0.01.

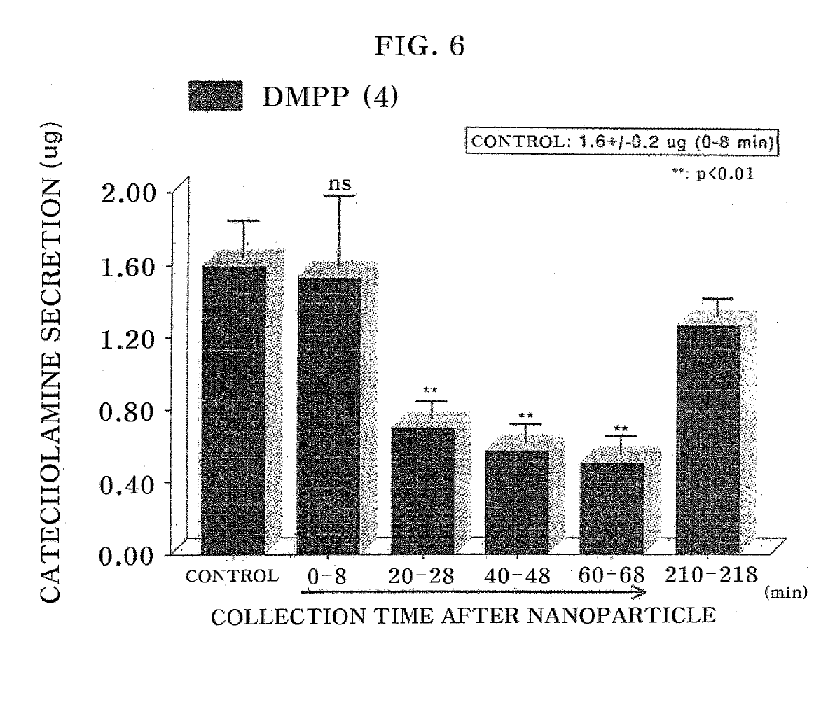

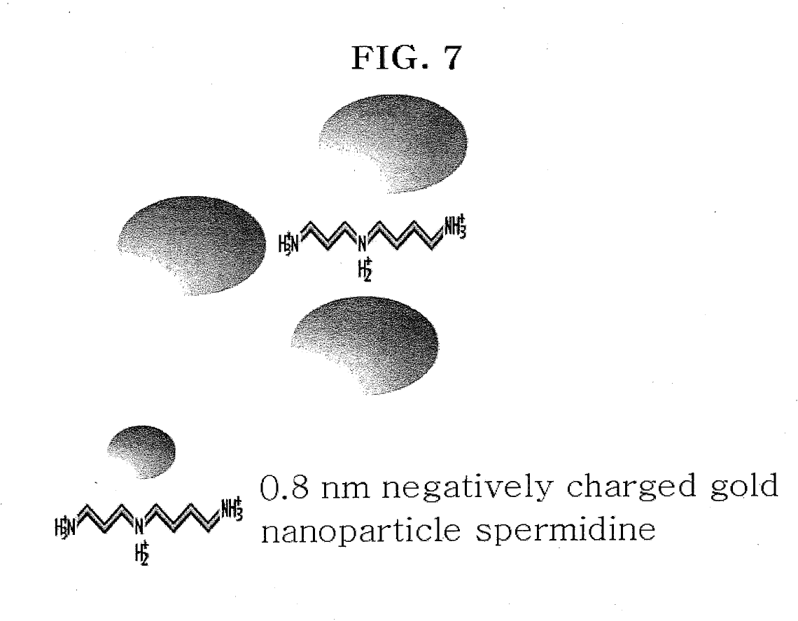

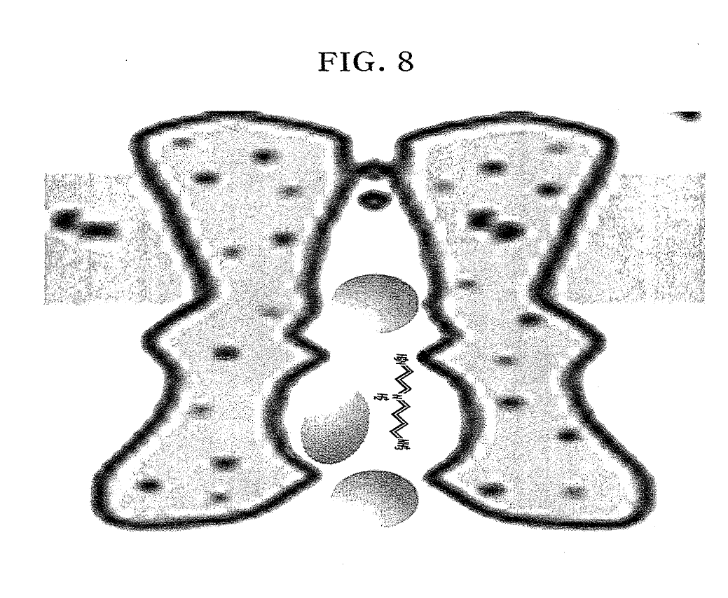

FIG. 6 illustrates the results of an experiment for the effect of the choline-nanoparticle complexes employing DMPP over time. When a single injection of DMPP was evoked in a rat adrenal gland, the receptor activation was confirmed after 15 minutes, and then when the choline-nanoparticle complexes were treated, it was confirmed that the catecholamine secretion was decreased to about 75% after 60 minutes. When DMPP was again treated after about 3.5 hours, it was confirmed that immediate reversal was achieved. *: P<0.01.

By binding gold nanoparticles to choline through ionic bonds, a drug having a stronger muscle relaxing effect obtained by blocking physically other ionic paths than prior muscle relaxants competitively inhibiting a receptor-ligand binding can be developed. Further, the complexes can be applied as a therapeutic agent for epilepsy.

The invention claimed is:

1. A method to achieve muscle relaxing effect or anti-atrial fibrillation effect, wherein gold nanoparticles complexes having an anion binds to choline or spermidine through ionic bonds migrate to an acetylcholine receptor and then the ionic bonds of the complexes are broken, so that the gold nanoparticles bind to the Cys loop; and when the reverse is administered, the gold-sulfur bonds are broken due to the electric current recovered to the receptor, and thus the gold nanoparticles are released ex vivo.

2. A method of claim 1, wherein the gold nanoparticles-choline complexes are injected intravenously into a patient on surgery, the complexes migrates to an acetylcholine receptor of a muscle and block the chemical and electrical neurotransmittance, respectively, and thus doubling the muscle relaxing effect.

3. A method of claim 1, wherein the gold nanoparticles-spermidine complexes are injected through cardiac catheterization into the atrial lumen, the complexes can penetrate into cell membrane and have a blocking effect of inward rectifying potassium channels in human atrial cells.

4. Muscle relaxant in form of gold nanoparticles-choline complexes which practice the method according to claim 1.

5. Anti-atrial fibrillation agent in form of gold nanoparticles-spermidine complexes which practice the method according to claim 1.

6. Muscle relaxant in form of gold nanoparticles-choline complexes which practice the method according to claim 2.

7. Anti-atrial fibrillation agent in form of gold nanoparticles-spermidine complexes which practice the method according to claim 3.
